SEQUENCE LISTING (13 sequences)

SEQ ID NO: 1 - protein, 126 aa, synthetic construct
  source 1..126: note = 2-B1 VHH
QVQLVESGGGLVQPGGSLTVSCAASGFSLDAYTIGWFRQAPGKEREGIICISRGGRITNYAHSVKDRFTISRDGAKNTVYLHMNSLKPEDTGAYYCAAVQRDPFICSGYTQSYDHWGQGTQVTVSS

SEQ ID NO: 2 - protein, 126 aa, synthetic construct
  source 1..126: note = 2-F3 VHH
QVKLEESGGGLVQPGGSLTVSCVASGFSLDAYTIGWFRQAPGKEREGIICISRGGRITNYAHSVKDRFTISRDGAKNMVYLHMNSLKPEDTGAYYCAAVQRDPFICSGYTQSYDLWGQGTQVTVSS

SEQ ID NO: 3 - protein, 126 aa, synthetic construct
  source 1..126: note = 2-C10 VHH
QVQLVESGGGLVQPGGSLRLSCAASGFSLDAYTIGWFRQAPGQEREGISCISRSGRITNYARSAKDRFTISRDNAKNTVYLEMKNLRPEDTGAYYCAAVEMDPFLCSGYTQSYDQWGQGTQVTVSS

SEQ ID NO: 4 - protein, 127 aa, synthetic construct
  source 1..127: note = 3-H6 VHH
QLKVVESGGGLVQPGESLRLSCAASGFTLGNYGVAWFRQAPGKEREGLSCITSGSTSVTHADSVKGRFSVSRDSDKKMVYLQMDSLKPEDTGIYYCAAADGGYVCSRNVNANEFDYWGQGTQVTVSS

SEQ ID NO: 5 - protein, 8 aa, synthetic construct
  source 1..8: note = CDR
GFSLDAYT

SEQ ID NO: 6 - protein, 8 aa, synthetic construct
  source 1..8: note = CDR
ISRGGRIT

SEQ ID NO: 7 - protein, 19 aa, synthetic construct
  source 1..19: note = CDR
AAVQRDPFICSGYTQSYDH

SEQ ID NO: 8 - protein, 19 aa, synthetic construct
  source 1..19: note = CDR
AAVQRDPFICSGYTQSYDL

SEQ ID NO: 9 - protein, 8 aa, synthetic construct
  source 1..8: note = CDR
ISRSGRIT

SEQ ID NO: 10 - protein, 19 aa, synthetic construct
  source 1..19: note = CDR
AAVEMDPFLCSGYTQSYDQ

SEQ ID NO: 11 - protein, 8 aa, synthetic construct
  source 1..8: note = CDR
GFTLGNYG

SEQ ID NO: 12 - protein, 8 aa, synthetic construct
  source 1..8: note = CDR
ITSGSTSV

SEQ ID NO: 13 - protein, 20 aa, synthetic construct
  source 1..20: note = CDR
AAADGGYVCSRNVNANEFDY

(19) United States
(12) Patent Application Publication
(10) Pub. No.: US 2026/0242474 A1
(43) Pub. Date: Aug. 20, 2026

(54) DEVELOPMENT OF NEW PD1 SINGLE DOMAIN ANTIBODY

(71) Applicant: PERSONGEN BIOTHERAPEUTICS (SUZHOU) CO., LTD., Suzhou, Jiangsu (CN)

(72) Inventors: Lin YANG, Suzhou, Jiangsu (CN); Fengtao YOU, Suzhou, Jiangsu (CN)

(21) Appl. No.: 18/722,961

(22) PCT Filed: Dec. 21, 2022
(86) PCT No.: PCT/CN2022/140768

§ 371 Date: Jun. 21, 2024

(30) Foreign Application Priority Data
Dec. 21, 2021 (CN) 202111571103.X

(51) Int. Cl. C07K 16/28; C12N 15/10; G01N 33/68

(52) U.S. Cl. CPC C07K 16/2818; C12N 15/1037; G01N 33/6845; C07K 2317/35; C07K 2317/52; C07K 2317/569; C07K 2317/76; C07K 2317/92

(57) ABSTRACT

A plurality of anti-PD-1 single domain antibodies are obtained by means of experimental screening, and a recombinant bivalent anti-PD-1 antibody is constructed. The anti-PD-1 single domain antibody obtained can efficiently bind to PD-1, can efficiently block the binding of PD-1/PD-L1.

### TECHNICAL FIELD

The present invention relates to the field of biomedicine. Specifically, the present invention relates to an anti-PD-1 single domain antibody.

### BACKGROUND

The specific immune response of T cells is triggered by the first stimulatory signal formed by the binding of T cell receptors with antigen peptide MHC complexes. Meanwhile, the intensity and amplitude of T cell-mediated immune response are regulated by immune checkpoint proteins, including co-inhibitory and co-stimulatory molecules. After more than 20 years of research, immune checkpoint molecules have been found to play a crucial role in regulating T cell immune responses. These molecules include Cytotoxic lymphocyte antigen-4 (CTLA-4), Programmed death-1 (PD-1), T cell immunoglobulin and mucin domain 3 (TIM-3), T cell Ig and ITIM domain (TIGIT), etc.

Under normal physiological conditions, the main function of immune checkpoints is to maintain self-tolerance and protect normal tissues from damage by the immune system. In the tumor microenvironment, due to prolonged exposure to tumor antigens, lymphocytes abnormally express certain co-inhibitory molecules, leading to dysfunction of tumor-specific T cells. A large amount of clinical data has shown that antibodies targeting co-inhibitory signals can enhance the response of tumor-specific T cells. Therefore, these immune checkpoint proteins have become potential targets for anti-tumor immunotherapy. Moreover, CAR-T cells can further improve the therapeutic effect on solid tumors by co-expressing PD1 antibodies.

However, the current performance of antibodies is still unsatisfactory. Therefore, there is a need in this field to develop new and excellent antibody drugs targeting the above-mentioned targets to meet clinical requirements.

Currently, most antibodies are derived from monoclonal antibodies and have limited blocking activity. Moreover, due to the large molecular weight of monoclonal antibodies, it is not easy for CAR-T cells to co-express PD1 antibodies at the same time.

Therefore, there is a need in this field to develop a novel PD1 nanobody.

### SUMMARY OF THE INVENTION

The purpose of the present invention is to provide a PD-1-targeting single domain antibody and use thereof.

In the first aspect of the present invention, it provides an anti-PD-1 single domain antibody, wherein the complementarity determination regions (CDRs) of the VHH chain of the anti-PD-1 single domain antibody is one or more selected from the group consisting of:
(1) CDR1 as shown in SEQ ID NO: 5, CDR2 as shown in SEQ ID NO: 9, and CDR3 as shown in SEQ ID NO: 10;
(2) CDR1 as shown in SEQ ID NO: 5, CDR2 as shown in SEQ ID NO: 6, and CDR3 as shown in SEQ ID NO: 8;
(3) CDR1 as shown in SEQ ID NO: 5, CDR2 as shown in SEQ ID NO: 6, and CDR3 as shown in SEQ ID NO: 7; and
(4) CDR1 as shown in SEQ ID NO: 11, CDR2 as shown in SEQ ID NO: 12, and CDR3 as shown in SEQ ID NO: 13.

In another preferred embodiment, the anti-PD-1 single domain antibody is capable of specifically binding to PD-1.

In another preferred embodiment, the anti-PD-1 single domain antibody is capable of blocking the binding between PD-1 and PD-L1.

In another preferred embodiment, any one of the amino acid sequences further comprises a derivative sequence that is optionally added, deleted, modified and/or substituted with at least one (such as 1-3, preferably 1-2, more preferably 1) amino acid and is capable of retaining the ability to specifically bind to PD-1.

In another preferred embodiment, the anti-PD-1 single domain antibody further comprises framework regions (FRs).

In another preferred embodiment, the CDR1, CDR2 and CDR3 are separated by framework regions FR1, FR2, FR3 and FR4 of the VHH chain.

In another preferred embodiment, the framework regions FR1, FR2, FR3 and FR4 comprise sequences derived from SEQ ID NO: 1, 2, 3 or 4.

In another preferred embodiment, the amino acid sequence of the VHH chain of the anti-PD-1 single domain antibody is selected from sequences shown in SEQ ID NOs: 1, 2, 3 and 4.

In another preferred embodiment, the anti-PD-1 single domain antibody includes a monomer, a bivalent (bivalent antibody), and/or a multivalent antibody.

In another preferred embodiment, the multivalent antibody comprises two or more (preferably 2, 3 or 4) VHH chains of the single domain antibody according to the first aspect of the present invention.

In another preferred embodiment, the anti-PD-1 single domain antibody is a bivalent.

In another preferred embodiment, the anti-PD-1 single domain antibody has a structure shown in Formula I from N-terminus to C-terminus:

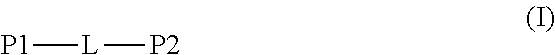

wherein,
“-” is a peptide bond;
L is a linker peptide;
P1 and P2 are each independently a VHH chain.

In another preferred embodiment, the sequence of L is (G4S)n, wherein n is a positive integer, such as 1, 2, 3, 4, 5 or 6, and preferably n=3.

In another preferred embodiment, P1 and P2 each independently comprises a sequence as shown in SEQ ID NO: 1, 2, 3 or 4.

In another preferred embodiment, P1 has an amino acid sequence as shown in SEQ ID NO: 3, and P2 has an amino acid sequence as shown in SEQ ID NO: 2.

In another preferred embodiment, P1 has an amino acid sequence as shown in SEQ ID NO: 3, and P2 has an amino acid sequence as shown in SEQ ID NO: 1.

In another preferred embodiment, P1 has an amino acid sequence as shown in SEQ ID NO: 2, and P2 has an amino acid sequence as shown in SEQ ID NO: 1.

In another preferred embodiment, the anti-PD-1 single domain antibody includes a humanized antibody, a camel antibody, a chimeric antibody.

In the second aspect of the present invention, it provides an anti-PD-1 antibody, which comprises one or more VHH chain(s) of the anti-PD-1 single domain antibody according to the first aspect of the present invention.

In another preferred embodiment, the amino acid sequence of the VHH chain of the anti-PD-1 single domain antibody is selected from sequences shown in SEQ ID NOs: 1, 2, 3 and 4.

In another preferred embodiment, the anti-PD-1 antibody includes a monomer, a bivalent (bivalent antibody), and/or a multivalent antibody.

In the third aspect of the present invention, it provides a polynucleotide encoding a protein selected from the group consisting of the anti-PD-1 single domain antibody of the first aspect of the present invention, and the antibody of the second aspect of the present invention.

In another preferred embodiment, the present invention relates to a nucleic acid molecule encoding the anti-PD-1 single domain antibody of the present invention. The nucleic acid molecule of the present invention may be RNA, DNA or cDNA.

In the fourth aspect of the present invention, it provides an expression vector comprising the polynucleotide of the third aspect of the present invention.

In another preferred embodiment, the expression vector is selected from the group consisting of DNA, RNA, a viral vector, a plasmid, a transposon, other gene transfer system, and a combination thereof.

In another preferred embodiment, the vector is a pcDNA3.4-hIgG1-Fc2 plasmid.

In the fifth aspect of the present invention, it provides a host cell comprising the expression vector of the fourth aspect of the present invention, or having the polynucleotide of the third aspect of the present invention integrated in its genome.

In another preferred embodiment, the host cell includes a prokaryotic cell or an eukaryotic cell.

In another preferred embodiment, the host cell is selected from the group consisting of Escherichia coli, a yeast cell, and a mammalian cell.

In another preferred embodiment, the host cell is a 293F cell.

In the sixth aspect of the present invention, it provides a method for producing an anti-PD-1 single domain antibody, which comprises the steps of:
(a) culturing the host cell of the fifth aspect of the present invention under conditions suitable for producing a single domain antibody, thereby obtaining a culture containing the anti-PD-1 single domain antibody;
(b) isolating or recovering the anti-PD-1 single domain antibody from the culture; and
(c) optionally, purifying and/or modifying the anti-PD-1 single domain antibody obtained in step (b).

In the seventh aspect of the present invention, it provides an immunoconjugate, which comprises:
(a) the anti-PD-1 single domain antibody of the first aspect of the present invention, or the anti-PD-1 antibody of the second aspect of the present invention; and
(b) a coupling moiety selected from the group consisting of a detectable label, a drug, a cytokine, a radionuclide, an enzyme, a gold nanoparticle/nanorod, a nanomagnetic particle, a viral coat protein or VLP, and a combination thereof.

In another preferred embodiment, the radionuclide comprises:
(i) a diagnostic isotope, which is selected from the group consisting of Tc-99m, Ga-68, F-18, I-123, I-125, I-131, In-111, Ga-67, Cu-64, Zr-89, C-11, Lu-177, Re-188, and a combination thereof; and/or
(ii) a therapeutic isotope, which is selected from the group consisting of Lu-177, Y-90, Ac-225, As-211, Bi-212, Bi-213, Cs-137, Cr-51, Co-60, Dy-165, Er-169, Fm-255, Au-198, Ho-166, I-125, I-131, Ir-192, Fe-59, Pb-212, Mo-99, Pd-103, P-32, K-42, Re-186, Re-188, Sm-153, Ra223, Ru-106, Na24, Sr89, Tb-149, Th-227, Xe-133 Yb-169, Yb-177, and a combination thereof.

In another preferred embodiment, the coupling moiety is a detectable label.

In another preferred embodiment, the coupling moiety is selected from the group consisting of a fluorescent or luminescent label, a radioactive label, MRI (magnetic resonance imaging) or CT (electronic computer X-ray tomography technique) contrast agent, or an enzyme capable of producing a detectable product, a radionuclide, a biotoxin, a cytokine (such as IL-2, etc.), an antibody, an Fc fragment of an antibody, an scFv fragment of an antibody, a gold nanoparticle/nanorod, a viral particle, a liposome, a nanomagnetic particle, a prodrug activating enzyme (such as DT-diaphorase (DTD) or biphenyl hydrolase-like protein (BPHL)), and a nanoparticle in any form.

In another preferred embodiment, the immunoconjugate comprises a multivalent (such as bivalent) VHH chains of the anti-PD-1 single domain antibody according to the first aspect of the present invention.

In another preferred embodiment, the multivalent refers that the amino acid sequence of the immunoconjugate contains multiple repeated VHH chains of the anti-PD-1 single domain antibody according to the first aspect of the present invention.

In the eighth aspect of the present invention, it provides a use of the anti-PD-1 single domain antibody of the first aspect of the present invention, the anti-PD-1 antibody of the second aspect of the present invention, or the immunoconjugate of the ninth aspect of the present invention for preparing:
(1) a drug for preventing and/or treating a disease related to PD-1 signaling pathway; and/or
(2) a reagent for detecting PD-1 signal.

In another preferred embodiment, the disease related to PD-1 signaling pathway includes a cancer or an autoimmune disease.

In another preferred embodiment, the cancer or tumor is selected from the group consisting of: hematological tumors, lymphoma, solid tumors, and a combination thereof.

In another preferred embodiment, the hematological tumor is selected from the group consisting of: acute myeloid leukemia (AML), multiple myeloma (MM), chronic lymphocytic leukemia (CLL), acute lymphocytic leukemia (ALL), diffuse large B cell lymphoma (DLBCL), and a combination thereof.

In another preferred embodiment, the lymphoma is selected from the group consisting of: Hodgkin lymphoma (HL), diffuse large B-cell lymphoma (DLBCL), follicular lymphoma (FL), chronic lymphocytic leukocyte (CLL), small lymphocytic lymphoma (SLL), marginal zone lymphoma (MZL), mantle cell lymphoma (MCL), Burkitt lymphoma (BL), and other complex B-cell non-Hodgkin lymphomas.

In another preferred embodiment, the solid tumor is selected from the group consisting of gastric cancer, gastric cancer peritoneal metastasis, liver cancer, kidney tumor, lung cancer, small intestinal cancer, bone cancer, prostate cancer, colorectal cancer, breast cancer, colorectal carcinoma, cervical cancer, ovarian cancer, lymphoma, nasopharyngeal cancer, adrenal tumor, bladder tumor, non-small cell lung cancer (NSCLC), brain glioma, endometrial cancer, testicular cancer, colorectal cancer, urinary tract tumor, thyroid cancer, and a combination thereof.

In another preferred embodiment, the reagent is a diagnostic reagent, and preferably the diagnostic reagent is a detection slide or a detection plate.

In another preferred embodiment, the diagnostic reagent is used for detecting PD-1 or a fragment thereof in a sample.

In the ninth aspect of the present invention, it provides a pharmaceutical composition, which comprises:
(a) the anti-PD-1 single domain antibody of the first aspect of the present invention, the anti-PD-1 antibody of the second aspect of the present invention, or the immunoconjugate of the seventh aspect of the present invention; and
(ii) a pharmaceutically acceptable carrier.

In the tenth aspect of the present invention, it provides a recombinant protein, which comprises:
(i) the anti-PD-1 single domain antibody of the first aspect of the present invention, or the anti-PD-1 antibody of the second aspect of the present invention; and
(ii) optionally, a tag sequence to assist expression and/or purification.

In another preferred embodiment, the tag sequence includes an Fc tag, an HA tag and a His tag.

In another preferred embodiment, the recombinant protein specifically binds to PD-1.

In the eleventh aspect of the present invention, it provides a kit comprising the anti-PD-1 single domain antibody of the first aspect of the present invention, the anti-PD-1 antibody of the second aspect of the present invention, or the immunoconjugate of the seventh aspect of the present invention.

In the twelfth aspect of the present invention, it provides a method of preventing and/or treating a disease related to PD-1 signaling pathway, which comprises a step of: administering the anti-PD-1 single domain antibody of the first aspect of the present invention, the anti-PD-1 antibody of the second aspect of the present invention, or the immunoconjugate of the seventh aspect of the present invention, to a subject in need thereof.

In another preferred embodiment, the subject includes a mammal, such as a human.

In another preferred embodiment, the disease related to PD-1 signaling pathway includes a cancer or an autoimmune disease.

In the thirteenth aspect of the present invention, it provides a method for detection of PD-1 or a fragment thereof in a sample in vitro, wherein the method comprises the steps of:
(1) contacting the sample with the anti-PD-1 single domain antibody of the first aspect of the present invention, the anti-PD-1 antibody of the second aspect of the present invention, or the immunoconjugate of the seventh aspect of the present invention in vitro; and
(2) detecting whether an antigen-antibody complex is formed, wherein the formation of the complex indicates the presence of PD-1 or a fragment thereof in the sample.

In another preferred embodiment, the serotype of PD-1 includes: PD-11, PD-12, PD-15, PD-16, PD-17, PD-18, and PD-19.

In another preferred embodiment, the serotype of PD-1 includes: PD-15, PD-18 and PD-19.

In another preferred embodiment, the detection includes a diagnostic detection and a non-diagnostic detection.

In the fourteenth aspect of the present invention, it provides a method for diagnosis of a disease related to PD-1 signaling pathway, which comprises the steps of:
(i) collecting a sample from a subject to be diagnosed, contacting the sample with the anti-PD-1 single domain antibody of the first aspect of the present invention, the anti-PD-1 antibody of the second aspect of the present invention, or the immunoconjugate of the seventh aspect of the present invention; and
(ii) detecting whether an antigen-antibody complex is formed, wherein the formation of the complex indicates that the subject suffers from a disease related to PD-1 signaling pathway.

In the fifteenth aspect of the present invention, it provides a method for preparing a recombinant polypeptide which is the anti-PD-1 single domain antibody of the first aspect of the present invention or the anti-PD-1 antibody of the second aspect of the present invention, wherein the method comprises the steps of:
(a) culturing the host cell according to the fifth aspect of the present invention under conditions suitable for expression; and
(b) isolating the recombinant polypeptide from the culture.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

The following drawings are used to illustrate specific embodiments of the present invention and are not intended to limit the scope of the present invention as defined by the claims.

FIG. 1 shows the results of PD1-Fc protein expression detected by SDS-PAGE. Lane 1 represents the size of PD1-Fc protein, and Lane M represents protein Marker.

FIG. 2 shows the flow cytometry detection result of the binding of PD1-Fc protein to PDL1-overexpressing cell line CHO.

FIG. 3 shows the ELISA detection results of the immunological potency of the immune serum.

FIG. 4 shows the alignment results of sequences from the phage display library.

FIG. 5 shows the results of antigen solid-phase panning, wherein a total of four different antibody sequences were screened.

FIG. 6 shows the cross-panning results of cell-based and solid-phase panning for antibodies.

FIG. 7 shows the binding of the screened PD1 single domain antibodies to CHO cells overexpressing PD1.

FIG. 8 shows the blocking activity of PD1 single domain antibodies (monovalent). Nivolumab serves as a positive control PD1 antibody, while LAB190417-2-B1, LAB190417-2-C10, LAB190417-2-F3, and LAB190417-3-H6 are four PD1 antibodies screened by flow cytometry.

FIG. 9 shows the blocking activity of PD1 single domain antibodies (bivalent). Nivolumab serves as a positive control PD1 antibody, while 2-C10-(G4S)3-2-F3, 2-C10-(G4S)3-2-B1, and 2-F3-(G4S)3-2-B1 are three constructed bivalent PD1 antibodies.

### DETAILED DESCRIPTION

After extensive and intensive research, the present inventors have successfully obtained multiple anti-PD-1 single-domain antibodies through large amount of screening. Specifically, the present invention used PD1-Fc antigen protein to immunize llamas, screened the immune single domain antibody gene library (phage display library) using phage display technology, and performed panning and identification to obtain single domain antibody genes targeting PD-1. Relevant experimental results indicate that the anti-PD-1 single-domain antibodies obtained by the present invention can effectively bind to PD-1 and exhibit excellent blocking activity against the PD-1/PD-L1 binding. On this basis, the present invention has been completed.

### Terms

As used herein, the terms “antibody of the present invention”, “antibody described in the present invention”, “anti-PD-1 single domain antibody of the present invention”, “anti-PD-1 single domain antibody described in the present invention”, “anti-PD-1 single domain antibody”, and “single domain antibody against PD-1” have the same meaning and can be used interchangeably to refer to a single domain antibody that specifically recognizes and binds to PD-1.

### Antibody

The term “antibody” or “immunoglobulin” as used herein refers to a heterotetrameric glycoprotein having the same structural feature of about 150,000 daltons consisting of two identical light chains (L) and two identical heavy chains (H). Each light chain is linked to a heavy chain by a covalent disulfide bond, and the numbers of disulfide bonds between the heavy chains of different immunoglobulin isoforms are different. Each heavy and light chain also has regularly spaced intrachain disulfide bonds. One end of each heavy chain has a variable region (VH) followed by a plurality of constant regions. There is a variable region (VL) at one end of each chain and a constant region at the other end; the constant region of the light chain corresponds to the first constant region of the heavy chain; the variable region of the light chain corresponds to the variable region of the heavy chain. An interface is formed between the variable regions of the light and heavy chains by particular amino acid residues.

As used herein, the terms “single domain antibody”, “VHH”, “nanobody”, “single domain antibody (sdAb, or nanobody)” have the same meaning and can be used interchangeably to refer to the heavy chain variable region of an antibody clone constructing a single domain antibody (VHH) composed of only one heavy chain variable region, which is the smallest antigen-binding fragment with complete function. Usually, the antibody with natural deletion of light chain and heavy chain constant region 1(CH1) is obtained first, and then the variable region of the antibody heavy chain is cloned to construct a single domain antibody (VHH) composed of only one heavy chain variable region.

As used herein, the term “variable” means that some certain portions of the variable region of an antibody differ in sequence and contribute to the binding and specificity of each particular antibody to its particular antigen. However, the variability is not evenly distributed throughout the antibody variable region. It is concentrated in three regions in the light and heavy chain variable regions called complementarity determining regions (CDRs) or hypervariable regions. The more conserved portions of the variable regions are referred as framework regions (FRs). The variable regions of the natural heavy and light chains each comprises four FR regions, which are in a substantially 3-sheet configuration, and are linked by three CDRs that form the linker ring and, in some cases, form a partial β-sheet structure. The CDRs in each chain stand close together through FR regions and form the antigen-binding site of the antibody together with the CDRs of the other chain (see Kabat et al., NIH Publ. No. 91-3242, Vol. I, 647-669 (1991)). Constant regions are not directly involved in the binding of the antibodies to the antigens, but they exhibit different effector functions, such as antibody-dependent cellular cytotoxicity involved in antibodies.

As known to those skilled in the art, an immunoconjugate and the fusion expression product includes: a conjugate formed by a drug, a toxin, a cytokine, a radionuclide, an enzyme and other diagnostic or therapeutic molecules connecting to the antibody or a fragment thereof. The present invention also comprises a cell surface marker or antigen binding to the nanobody against PD-1 or a fragment thereof.

As used herein, the terms “heavy chain variable region” and “VH” can be used interchangeably.

As used herein, the terms “variable region” and “complementarity determine region (CDR)” can be used interchangeably.

In a preferred embodiment of the present invention, the heavy chain variable region of the antibody comprises three complementarity determining regions, CDR1, CDR2, and CDR3.

In a preferred embodiment of the present invention, the heavy chain of the antibody comprises the above-mentioned heavy chain variable region and a heavy chain constant region.

In the present invention, the terms “the antibody of the present invention”, “the protein of the present invention”, or “the polypeptide of the present invention” can be used interchangeably and all refer to an polypeptide specifically binding to PD-1, e.g., a protein or polypeptide with a heavy chain variable region. They can contain or do not contain starting methionine.

The invention also provides other proteins or fusion expression products comprising the antibody of the present invention. Specifically, the present invention includes any protein or protein conjugate and fusion expression product (i.e., immunoconjugate and fusion expression product) having a heavy chain containing variable regions, as long as the variable region is the same as or has at least 90% homology, preferably at least 95% homology with the variable region of the heavy chain of the antibody of the present invention.

The terms “specific binding”, “selective binding”, “selectively bind”, and “specifically bind”, refer to the binding of an antibody to an epitope on a predetermined antigen. Usually, an antibody has an affinity (KD) that is approximately less than 10⁻⁷M, such as approximately less than 10⁻⁹M, 10⁻⁹M or 10⁻¹⁰M or less.

In general, the antigen-binding properties of an antibody can be described by three specific regions located in the heavy chain variable region, referring as variable regions (CDRs), and separated into four framework regions (FRs). The sequences of four FRs amino acids are relatively conservative and do not directly participate in the binding reaction. A cyclic structure is formed by these CDRs which are close to each other in the spatial structure by the β-sheets formed by the FRs between them, and the CDRs on the heavy chains and the CDRs on the corresponding light chains constitute the antigen-binding sites of the antibody. The amino acid sequence of the same type of antibody can be used to determine which amino acids have constituted the FR or CDR regions.

The variable regions of the heavy chain of the antibody of the present invention are of particular interest because at least part of them involves binding antigens. Therefore, the present invention includes molecules with heavy chain variable regions of antibodies with CDRs, as long as their CDRs have more than 90% (preferably more than 95%, most preferably more than 98%) homology with the CDRs identified here.

The present invention includes not only intact antibodies, but also immunologically active fragments of antibody fragments or fusion proteins formed by antibodies and other sequences. Therefore, the present invention also includes fragments, derivatives and analogs of the antibodies.

As used herein, the terms “fragment”, “derivative” and “analog” refer to a polypeptide basically maintaining the same biological function or activity of the antibody of the present invention. The polypeptide fragment, derivative or analog of the present invention may be (i) a polypeptide with one or more conservative or non-conservative amino acid residues (preferably the conservative amino acid residues) being substituted, while such substituted amino acid residues may or may not be encoded by genetic code, or (ii) a polypeptide having substituted group(s) in one or more amino acid residues, or (iii) a polypeptide formed by fusion of the matured polypeptide with another compound (such as the compound that prolongs the half-life of the polypeptide, such as polyethylene glycol), or (iv) a polypeptide formed with additional amino acid sequence fused to said polypeptide sequence (such as, leader sequence, secretion sequence, or a sequence or a protein sequence used to purify the polypeptide, or a fusion protein formed with 6His tag). According to the teaching of the present application, these fragments, derivatives, and analogs are within the scope commonly known by those skilled in the art.

The antibody of the present invention refers to a polypeptide having PD-1 protein binding activity and comprising the above-mentioned CDR regions. The term also includes variant forms of polypeptides comprising the CDR regions described above that have the same function as the antibody of the present invention. These variant forms include, but are not limited to, deletion insertion and/or substitution of one or more amino acids (typically 1-50, preferably 1-30, more preferably 1-20, most preferably 1-10), and addition of one or several amino acids (typically at most 20, preferably at most 10, more preferably at most 5) at the C-terminus and/or N-terminus. For example, in the art, the protein's functions are usually unchanged when an amino acids is substituted by a similar or analogous one. Also, for example, the addition of one or several amino acids at the C-terminus and/or the N-terminus will not normally alter the function of the protein. The term also includes active fragments and active derivatives of the antibody of the present invention.

The variant forms of the antibody include homologous sequences, conserved variants, allelic variants, natural mutants, induced mutants, proteins encoded by a DNA capable of hybridizing to the coding DNA of the antibody of the present invention under high or low stringency conditions, and a polypeptide or protein obtained using an antiserum against the antibody of the present invention.

The present invention also provides other polypeptides, such as fusion proteins containing antibodies or fragments thereof. In addition to the almost full-length polypeptide, the present invention also includes fragments of the human antibody of the present invention. Typically, the fragment has at least about 50 contiguous amino acids, preferably at least about 50 contiguous amino acids, more preferably at least about 80 contiguous amino acids, and most preferably at least about 100 contiguous amino acids of the antibody of the present invention.

In the present invention, “the conservative variant of the antibody of the present invention” refers to a polypeptide that comprises at most 10, preferably at most 8, more preferably at most 5, most preferably at most 3 amino acids replaced by amino acids with the same or similar properties compared with the amino acid sequence of the antibody of the present invention. These conservatively variant polypeptides are preferably produced by amino acid substitution according to Table A.

TABLE A

| Initial | Representative | Preferred |
|---|---|---|
| residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides a polynucleotide molecule encoding the antibody or a fragment thereof or a fusion protein thereof. The polynucleotides of the present invention can be in a form of DNA or RNA. DNA forms include cDNA, genomic DNA, or synthetic DNA. DNA can be singe-stranded or double-stranded. DNA can be the coding strand or the non-coding strand.

The polynucleotides encoding the mature polypeptides of the present invention comprise coding sequences encoding only the mature polypeptide; coding sequences of the mature polypeptide and various additional coding sequences; coding sequences (and optionality additional coding sequences) of the mature polypeptide, and non-coding sequences.

The term “polynucleotide encoding a polypeptide” may include a polynucleotide that encodes the polypeptide, or a polynucleotide that also includes additional coding and/or non-coding sequences.

The present invention also relates to polynucleotides that hybridize to the sequences as described above and having at least 50%, preferably at least 70%, more preferably at least 80% identical between the two sequences. In particular, the present invention relates to polynucleotides that can hybridize to the polynucleotides of the present invention under stringent conditions. In the present invention, “stringent conditions” means: (1) hybridization and elution at lower ionic strength and higher temperature, such as 0.2×SSC, 0.1% SDS, 60° C.; or (2) hybridization adding a denaturant, such as 50% (v/v) formamide, 0.1% calf serum/0.1% Ficoll, 42° C., or the like; or (3) hybridization only occurs when the identity between the two sequences is at least 90%, more preferably 95% or more. And the polypeptide encoded by the hybridizable polynucleotide has the same biological function and activity as the mature polypeptide.

The whole length of the nucleotide sequence or the fragment thereof of the antibody of the present invention can be obtained via PCR amplification, recombinant method or artificial synthesis. One feasible method is to synthesize relevant sequences by artificial method, especially when the fragment is short in length. Usually, several small fragments are synthesized first, and then are linked together to obtain a fragment with a long sequence. In addition, the sequence coding the heavy chain and the expression tag (e.g. 6His) can be fused together to form a fusion protein.

Once a relevant sequence is obtained, the relevant sequence can be obtained in bulk using a recombination method. This is usually carried out by cloning the sequence into a vector, transforming a cell with the vector, and then separating the relevant sequence from the proliferated host cell by conventional methods. The biomolecules (nucleic acids, proteins, etc.) involved in the present invention include biomolecules that exist in an isolated form.

At present, DNA sequences encoding the protein of the invention (or fragments thereof, or derivatives thereof) can be completely obtained by chemical synthesis. The DNA sequence can then be introduced into a variety of existing DNA molecules (or vectors) and cells known in the art. In addition, mutations can also be introduced into the protein sequences of the present invention by chemical synthesis.

The present invention further relates to a vector comprising said suitable DNA sequence and a suitable promoter or a control sequence. These vectors can be used to transform suitable host cells to enable them to express protein.

The host cell can be a prokaryotic cell, such as a bacterial cell; or a lower eukaryotic cell, such as a yeast cell; or a higher eukaryotic cell, such as a mammalian cell. Representative examples are: Escherichia coli, streptomactinus, bacterial cells of Salmonella typhimurium; fungal cells such as yeast; insect cells of Drosophila S2 or SF9; animal cells of CHO, COS7, 293 cells, etc.

Transformation of a host cell with a recombinant DNA can be carried out by conventional techniques well known to those skilled in the art. When the host is a prokaryotic organism such as Escherichia coli, competent cells that can absorb DNA can be harvested after the exponential growth phase then treated with the CaCl₂) method, and the steps used are well known in the art. Another method is to use MgCl₂. If necessary, the transformation can also be carried out by electroporation. When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the polypeptide encoded by the gene of the present invention. Depending on the host cell used, the medium used during the culture can be selected from various conventional mediums. The culture is carried out under conditions suitable for the growth of the host cell. When the host cell has grown to an appropriate cell density, a suitable method (such as temperature conversion or chemical induction) is used to induce the selected promoter, and the cell is cultured for another period of time.

The recombinant polypeptide described in the above method can be expressed intracellularly or on the cell membrane, or be secreted out of the cell. If desired, recombinant proteins can be isolated and purified by various separation methods utilizing their physical, chemical, and other properties. These methods are well known to those skilled in the art. Examples of such methods include, but are not limited to, conventional renaturation treatments, treatment with a protein precipitant (salting-out method), centrifugation, osmosis cell disruption, super-treatment, ultracentrifugation, molecular sieve chromatography (gel filtration), adsorption chromatography, ion-exchange chromatography, high performance liquid chromatography (HPLC) and various other liquid chromatography techniques and combinations of these methods.

The antibody of the present invention can be used alone, or can be combined or coupled with a detectable label (for diagnostic purposes), a therapeutic agent, a PK (protein kinase) modified moiety, or any combination of these substances.

Detectable labels for diagnostic purposes include, but are not limited to: fluorescent or luminescent labels, radioactive labels, MRI (magnetic resonance imaging) or CT (electronic computer X-ray tomography technique) contrast agents, or enzymes capable of producing detectable products.

A therapeutic agent that can be combined or coupled with the antibody of the present invention includes, but is not limited: 1. a radionuclide; 2. a biological toxin; 3. a cytokine such as IL-2, etc; 4. a gold nanoparticle/nanorod; 5. a viral particle; 6. a liposome; 7. a magnetic nanoparticle; 8. a prodrug-activating enzyme (e.g., DT-diaphorase (DTD) or biphenyl hydrolase-like protein (BPHL)).

### PD-1

As used herein, the terms “PD-1” and “Programmed Death Receptor 1” both refer to the PD-1 immune checkpoint receptor, which can restrain T-cell activity and limit autoimmunity during peripheral tissue inflammatory responses. PD-1 is a primary mechanism for immune resistance in the tumor microenvironment. T-cell activation induces the expression of PD-1. Upon binding to one of PD-1 ligands, the phosphatase SHP2 involved in T-cell activation is inhibited. Simultaneously, since PD-1 binding inhibits the TCR stop signal, this signal pathway alters the duration time of interaction between T cells and antigen-presenting cells or between T cells and target cells. PD-1 is highly expressed on T regulatory (Treg) cells, and the proliferation of which is enhanced in the presence of PD-1 ligands. Given that many tumors highly express infiltrative Tregs, which may further suppress the immune responses of effector cells, blocking the PD-1 signal pathway will reduce and/or inhibit Treg cell activity within tumors, thus enhancing anti-tumor immunity.

PD-1 has two ligands, PD-L1 and PD-L2.

PD-1 is expressed in a majority of tumor-infiltrating lymphocytes (TILs) from various types of tumors. CD4+ TILs with high PD-1 expression are predominantly found among CD4+ Tregs in tumor tissues. CD8+ TILs with high PD-1 expression show an exhausted or dysfunctional state. Compared to PD-1-TILs, PD-1+ TILs in melanoma secrete fewer cytokines. Similar to PD-1 which is overexpressed on many TILs in tumors, PD-1 ligands are also highly expressed on the surface of diverse tumor cells. PD-L1, the main ligand of PD-1, is expressed on solid tumor cells, enabling mouse tumor cells with high PD-L1 expression to suppress T-cell mediated anti-tumor immune responses. Indeed, these findings provide a basis for interrupting the PD-1 signal pathway to boost the function of anti-tumor effector cells in the tumor microenvironment. Immunohistochemistry (IHC) and flow cytometry analyses reveal a certain level of constitutive high expression of PD-1 ligands in various human tumors. The expression pattern of PD-1 ligand largely determines the feasibility of immune therapy strategies targeting this pathway, since PD-1 ligands primarily exert immunosuppressive effects in the tumor microenvironment, and the binding of PD-1 with its ligands, PD-L1 or PD-L2, solely inhibits lymphocyte functions.

### Pharmaceutical Composition

The present invention further provides a composition. Preferably, the composition is a pharmaceutical composition comprising the antibody, or an active fragment or a fusion protein thereof, and a pharmaceutically acceptable carrier. In general, these substances may be formulated in a non-toxic, inert and pharmaceutically acceptable aqueous carrier medium, wherein the pH is generally about 5-8, preferably, pH is about 6-8, though the pH value may be varied depending on the nature of the substances to be formulated and the condition to be treated. The formulated pharmaceutical composition may be administered by conventional routes, including (but not limited to): intraperitoneal, intravenous, or topical administration.

The pharmaceutical composition according to the present invention comprises a safe and effective amount (e.g., 0.001-99 wt %, preferably 0.01-90 wt %, more preferably 0.1-80 wt %) of the antibody according to the present invention (or a conjugate thereof) and a pharmaceutically acceptable carrier or excipient. Such carriers include (but are not limited to): saline, buffers, glucose, water, glycerol, ethanol, and a combination thereof. Pharmaceutical preparations should correspond to the administration modes. The pharmaceutical composition according to the present invention can be prepared in the form of an injection, for example, by a conventional method using physiological saline or an aqueous solution containing glucose and other adjuvants. A pharmaceutical composition, for example, an injection and a solution, should be prepared under aseptic conditions. The administration amount of an active ingredient is a therapeutically effective amount, for example, about 10 μg per kilogram of body weight to about 50 mg per kilogram of body weight daily. In addition, the polypeptide according to the present invention may also be used in combination with an additional therapeutic agent.

When a pharmaceutical composition is used, a safe and effective amount of immunoconjugate is administered to a mammal, wherein the safe and effective amount is generally at least about 10 μg per kilogram of body weight, and in most cases, no more than about 50 mg per kilogram of body weight, preferably, the amount is from about 10 μg per kilogram of body weight to about 10 mg per kilogram of body weight. Of course, a specific amount should also depend on the factors such as administration route and physical conditions of a patient, which fall into the skills of skilled physicians.

### Anti-PD-1 Single Domain Antibody

The present invention provides an anti-PD-1 single domain antibody which is capable of specifically binding to PD-1.

The complementarity determination regions (CDRs) of a VHH chain of the anti-PD-1 single domain antibody according to the present invention is one or more selected from the group consisting of:
(1) CDR1 as shown in SEQ ID NO: 5, CDR2 as shown in SEQ ID NO: 9, and CDR3 as shown in SEQ ID NO: 10;
(2) CDR1 as shown in SEQ ID NO: 5, CDR2 as shown in SEQ ID NO: 6, and CDR3 as shown in SEQ ID NO: 8;
(3) CDR1 as shown in SEQ ID NO: 5, CDR2 as shown in SEQ ID NO: 6, and CDR3 as shown in SEQ ID NO: 7; and
(4) CDR1 as shown in SEQ ID NO: 11, CDR2 as shown in SEQ ID NO: 12, and CDR3 as shown in SEQ ID NO: 13.

In one preferred embodiment of the present invention, the anti-PD-1 single domain antibody comprises one or more VHH chain(s) which has an amino acid sequence shown in SEQ ID NO: 1, 2, 3 or 4.

In the present invention, the anti-PD-1 single domain antibody includes monomer, bivalent (bivalent antibody), tetravalent (tetravalent antibody), and/or multivalent (multivalent antibody). The multivalent antibody comprises two or more (preferably 2, 3 or 4) the above VHH chains or single domain antibodies.

In one preferred embodiment of the present invention, the anti-PD-1 single domain antibody is a bivalent.

The bivalent (bivalent antibody) of the present invention has a structure as shown in Formula I:

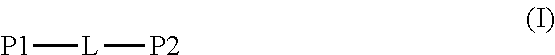

wherein,
“-” is a peptide bond; L is a linker peptide, and P1 and P2 are VHH chains.

In one preferred embodiment of the present invention, the sequence of L is (G4S)n, wherein n is a positive integer, such as 1, 2, 3, 4, 5 or 6, and preferably n=3.

In another preferred embodiment, P1 and P2 each independently comprises a sequence as shown in SEQ ID NO: 1, 2, 3 or 4.

In another preferred embodiment, P1 has an amino acid sequence as shown in SEQ ID NO: 3, and P2 has an amino acid sequence as shown in SEQ ID NO: 2.

In another preferred embodiment, P1 has an amino acid sequence as shown in SEQ ID NO: 3, and P2 has an amino acid sequence as shown in SEQ ID NO: 1.

In another preferred embodiment, P1 has an amino acid sequence as shown in SEQ ID NO: 2, and P2 has an amino acid sequence as shown in SEQ ID NO: 1.

### Detection Method

The present invention also relates to a method for detecting PD-1. The steps of the method are roughly as follows: obtaining a cell and/or tissue sample; dissolving the sample in a medium; and detecting the level of PD-1 in the dissolved sample.

In the detection method of the present invention, the sample used is not particularly limited, and a representative example is a cell-containing sample present in a cell preservation solution.

### Kit

The present invention also provides a kit containing the antibody (or a fragment thereof) or the detection plate of the present invention. In a preferred embodiment of the present invention, the kit further comprises a container, an instruction, and a buffer, etc.

The present invention also provides a detection kit for detecting the PD-1 level, which comprises an antibody that recognizes PD-1 proteins, a lysis medium for dissolving a sample, common reagents and buffers required for detection, such as various buffers, detection labels, detection substrates, etc. The detection kit may be an in vitro diagnostic device.

### Application

As described above, the antibody of the present invention has a wide range of biological application value and clinical application value, and its application relates to the diagnosis and treatment, basic medical research, biological research and other fields of the PD-1 related diseases. One preferred application is for clinical diagnosis, prevention and treatment for PD-1 signal pathway related diseases.

### Main Advantages of the Present Invention Include:

1) The antibody of the present invention can specifically bind to PD-1 and has high neutralizing activity against PD-1;
2) The antibody of the present invention can efficiently block the binding of PD-1/PD-L1; The PD1 nanobody of the present invention has the advantages of small molecular weight, fast tissue penetration, high solubility and stability, high antigen binding specificity, low immunogenicity, etc., and makes it easier for CAR-T cells to co-express PD1 antibody. The PD1 nanobody of the present invention can be used for cancer treatment.

The invention is further illustrated below in conjunction with specific embodiments. It should be understood that the examples are not intended to limit the scope of the invention. The experimental methods in the following examples which do not specify the specific conditions are usually in accordance with conventional conditions, such as conditions described in Sambrook et al., Molecular Cloning: Laboratory Manual (New York: Cold Spring Harbor Laboratory Press, 1989), or in accordance with the conditions recommended by the manufacturer. Unless otherwise stated, percentages and parts are calculated by weight.

### Experimental Reagents, Consumables and Equipment:

Reagents: Agar (Sigma, CAT #A1296); Peptone (Sigma, CAT #93926); Yeast Extract (OXOID, CAT #: LP0021); Sodium Chloride (Aladdin, CAT #: C111533); Potassium Chloride (Aladdin, CAT #: P112133); Magnesium Sulfate (Sinopharm, CAT #: 10013018); Magnesium Chloride (Sinopharm, CAT #: 10012818); Glucose (Sangon, CAT #: GT1991); SfiI (NEB, CAT #: R0123L); T4 DNA Ligase (TaKaRa, CAT #: 2011A); PrimeScript™ II 1st Strand cDNA Synthesis Kit (TaKaRa, CAT #: 6210B); NuHi power Mix (NUHIGH Bio, CAT #: NH9303); 3M Sodium Acetate (pH 5.2-6) (Sigma, CAT #: 126-96-5); DNA Fragment Recovery Kit (TakaRa, CAT #: 9761); Gel Recovery Kit (Qiagen, CAT #: 28706); Tiangen Plasmid Extraction Kit (Tiangen, CAT #: DP117); PmeI Restriction Enzyme (NEB, CAT #: R0560L); HRP-M13 (Sino Biological, CAT #: 11973-MM05); PE-anti-Human IgG (eBioscience, Cat #: 12-4998-82); Rabbit anti-Llama IgG (H+L) Secondary Antibody [HRP](Novus, CAT #NBP1-75095); SS320 Competent Cells (iCarTab); pComF Phage Display Vector (iCarTab).

Consumables: 50 mL Falcon Tubes (Corning, CAT #352070); Electroporation Cuvettes (Bio-Rad 0.2 cm); RNase-free 1.5 ml EP Tubes (QSP, CAT #: 509-GRD-Q); 200 μL RNase-free PCR Tubes (Axygen, PCR-02D-C); T125 Flask (Corning, CAT #431143).

Equipment: Electroporator (Eppendorf Multiporator); Centrifuge (Xiangyi H1650R); Constant Temperature Incubator (Shanghai Jinghong DNP-9052); Constant Temperature Shaking Incubator (Langyue DZ-85A); Super Clean Bench (Sujing Antai SW-CJ-1FD); PCR (Applied Biosystems ABI2720); Biological Safety Cabinet (Haier HR40-IIA2); Flow Cytometer (Thermo Attune Nxt flow cytometer).

### Experimental Methods

### 1. PD1-Fc Antigen Preparation

1) Sequence of the extracellular domain of PD1 (residues 25-167) was generated by gene synthesis, followed by attachment of a human IgG1 Fc tag at the C-terminus. The construct was subcloned into a eukaryotic expression vector to form the antigen expression vector.
2) The prepared PD1-Fc protein expression vectors were subjected to large-scale plasmid extraction, then transfected into 293 cells. Cells were continuously cultured for 8 days and centrifugated to collect the supernatant. The supernatant was filtered through a 0.45 μm filter membrane, and the filtrate was transferred to a sterile centrifuge tube. Antibody purification was performed using a Protein A column.

### 2. Antigen Immunization of Llama

The above prepared antigen was used to immunize a llama through subcutaneous multi-point injection for a total of five times. The immunization schedule is shown in Table 1 below.

TABLE 1
Llama Immunization Schedule

| | |
|---|---|
| Primary | 5 mL blood was collected as negative serum before |
| immunization | immunization; adjuvant was mixed with antigen (1 mg) |
| | at 1:1, emulsified, and subcutaneously injected at |
| | multiple points. |
| Second | Antigen (2 mg) was mixed with antigen at 1:1, |
| immunization | emulsified, and subcutaneously injected at multiple |
| | points. |
| Third | Antigen (2 mg) was mixed with antigen at 1:1, |
| immunization | emulsified, and subcutaneously injected at multiple |
| | points. |
| Fourth | Antigen (2 mg) was mixed with antigen at 1:1, |
| immunization | emulsified, and subcutaneously injected at multiple |
| | points. |
| Fifth | Antigen (2 mg) was mixed with antigen at 1:1, |
| immunization | emulsified, and subcutaneously injected at multiple |
| | points. |
| | Serum was sampled for titer test one week after the |
| | fifth immunization. |
| Boost | Antigen (2 mg), intravenous injection via jugular vein |
| immunization | |
| | 100 mL peripheral blood was sampled three days after |
| | boost immunization for construction of phage display |
| | library. |

### 3. Detection of Immunological Potency

1) 5 ml of peripheral blood was collected, and the centrifuge tube containing the blood sample was placed in an incubator at 37° C. for 1 hour. Afterward, the blood sample was transferred to 4° C. overnight.
2) The serum was transferred to a new sterile centrifuge tube and centrifuged at 5000 rpm for 20 minutes. The immunological potency was then detected by ELISA.

### 4. PBMC Isolation

100 ml of peripheral blood was collected, and PBMCs were isolated using lymphocyte separation medium.

RNA was extracted and subjected to reverse transcription using the PrimeScript™ II 1st Strand cDNA Synthesis Kit to prepare cDNA. The method was as follows:

Reaction mixture MIX1 shown in Table 2 was prepared in a 200 μL PCR tube:

TABLE 2

| Reagent | Quantity | |
|---|---|---|
| Oligo dT Primer (50 μM) | 8 | μL |
| dNTP Mixture (10 mM each) | 8 | μL |
| Total RNA sample | 20 | μg |

| | |
|---|---|
| RNase-Free Water | Up to 80 uL |

After incubation at 65° C. for 5 minutes, the mixture was rapidly cooled on ice.

The reaction solution shown in Table 3 was prepared in the same PCR tube as described above:

TABLE 3

| Reagent | Quantity | |
|---|---|---|
| The above denatured reaction mixture | 80 | μL |
| 5 × PrimeScript II Buffer | 32 | μL |
| RNase Inhibitor (40 U/μL) | 4 | μL |
| PrimeScript II RTase (200 U/μL) | 8 | μL |
| RNase-Free Water | 36 | μL |

After thorough mixing, 80 μL of the solution was dispensed into each tube and placed in a PCR instrument at 42° C. for 1 hour, followed by heat-inactivation at 70° C. for 15 minutes. Finally, the cDNA samples were stored on ice or at −20° C. for long-term preservation.

### 5. Amplification of VHH Fragments

The reaction system of the first round PCR was prepared according to Table 4 (50 L/tube).

TABLE 4

| Component | Quantity |
|---|---|
| Forward primer (5 μM) | 2 |
| Reverse primer (5 μM) | 1 |
| NuHi Power mix (2×) | 25 |
| cDNA template | 2 |
| Sterile water | 20 |

After the preparation of PCR reaction system, the PCR instrument was set up according to the procedure shown in Table 5:

TABLE 5

| | | | | Number |
|---|---|---|---|---|
| Program | Temperature | Time | | of cycles |
| Pre-denaturation | 95° C. | 10 | min | |
| Denaturation | 95° C. | 15 | S | 30X |
| Annealing | 55° C. | 30 | S | |
| Extension | 68° C. | 1 | min | |
| Final extension | 68° C. | 10 | min | |

Electrophoresis analysis on PCR products was performed by using 1% agarose, and fragments with a molecular weight of approximately 750 bp were isolated. PCR products were recovered by a gel recovery kit and subjected to the determination of concentration by NanoDrop.

The reaction system of the second round PCR was prepared according to Table 6 (50 μL/tube).

TABLE 6

| Component | Quantity |
|---|---|
| 2^{nd} F-primer | 2 |
| 2^{nd} R-primer | 2 |
| NuHi Power mix (2×) | 25 |
| Products recovered from the first round PCR | 200 ng |
| Sterile water | Add to 50 μL |

After the preparation of PCR reaction system, the PCR instrument was set up according to the procedure in Table 7:

TABLE 7

| | | | | Number |
|---|---|---|---|---|
| Program | Temperature | Time | | of cycles |
| Pre-denaturation | 95° C. | 10 | min | |
| Denaturation | 95° C. | 15 | S | 25X |
| Annealing | 55° C. | 30 | S | |
| Extension | 68° C. | 1 | min | |
| Final extension | 68° C. | 10 | min | |

Electrophoresis analysis on PCR products was performed by using 1% agarose, and fragments with a molecular weight of approximately 750 bp were isolated. PCR products were recovered by a gel recovery kit and subjected to the determination of concentration by NanoDrop.

### 6. Construction of Phage Display Library

### Construction of Phage Display Vector:

1) The pCom F vector and the previously obtained recovered PCR gel product of VHH were digested with SfiI at 50° C. overnight, respectively.
2) The pCom F vector fragment was isolated using 1% agarose gel. The gel of 5000 bp vector fragment was excised and recovered. Meanwhile, the PCR-digested product was purified using DNA Fragment Recovery Kit, and subjected to the determination of concentration by using NanoDrop.
3) The digested pCom F vector and VHH fragment were ligated using T4 ligase at 16° C. overnight.

Electrotransformation of phage ligation product into E. coli:
1) Electroporation cuvettes, ligation product, and electrotransformation competent cells were placed on ice for pre-cooling;
2) The pre-cooled library ligation product was added to the electrotransformation competent cells, kept on ice for 1 minute, and 70 μL of DNA/competent cell mixture was added to each electroporation cuvette, which was then placed on ice;
3) Electrotransformation was performed at 2500V for 5 ms;
4) After electroporation, cells were immediately resuspended by adding SOC culture medium equilibrated to room temperature, and cultured in a shaker at 37° C. for 1 hour.
5) 15 mL of the bacterial suspension was directly used for phage rescue, while the remaining 5 mL of the electroporation product was mixed with an equal volume of 50% glycerol, thoroughly mixed, and stored at −80° C.
6) Additionally, 20 μL of the bacterial suspension was diluted in 980 μL of 2YT culture medium, and 100 μL of the diluted product was further diluted in 900 μL of 2YT culture medium. 50 μL of the secondarily diluted product was evenly spread on a LB plate containing ampicillin and cultured at 37° C. overnight.
7) The next day, the plate was taken out to count the number of clones produced by each ligation and calculate the library capacity.
8) Simultaneously, 20 monoclonal colonies from the plate were picked and cultured in 2YT culture medium containing ampicillin at 37° C. for approximately 6-8 hours. The bacterial suspension was then subjected to sequencing by using universal primer M13R, in order to determine the diversity of the library.

### 7. Selection of the Phage Display Library

The steps for resuscitation and rescue of the phage display library and phage precipitation were as follows:
1) The electrotransformation product was diluted with 2YT to adjust the OD600 to approximately 0.2. Ampicillin with a final concentration of 100 μg/mL was added, and the mixture was incubated in a constant temperature shaker at 37° C. and 225 rpm until the OD600 reached 0.5;
2) M13KO7 was added. After thorough mixing, the mixture was allowed to stand at 37° C. for 30 minutes and then cultured at 37° C. and 225 rpm for 1 hour.
3)

$$Volume of M 13 KO 7 = 10 \times Volume \times OD 600 \times 5 \times 108 / Titer of M 13 KO 7$$

4) The bacterial suspension was centrifuged at 6000 rpm for 10 minutes and resuspended in 2YT-AK medium. It was then cultured overnight at 25° C. and 200 rpm;
5) The bacterial suspension was centrifuged at 10000 rpm for 15 minutes;
6) The precipitate was discarded, and the supernatant was transferred to a new centrifuge tube. PEG/NaCl with a volume of ⅕ bacterial suspension was added to the tube, mixed thoroughly, and the mixture was placed at 4° C. for 2 hours.
7) The precipitated phage supernatant was centrifuged at 10000 rpm, 4° C., for 30 minutes. The supernatant was discarded, and the precipitate (phage) in each 50 ml centrifuge tube was resuspended in 1 ml of sterile PBS.
8) The resuspended phage was transferred to a 1.5 mL EP tube and centrifuged at 12000 g, 4° C., for 5 minutes.
9) The supernatant was transferred to a new 1.5 ml EP tube, and 250 μl of PEG/NaCl was added to each tube. After thorough mixing, the tubes were placed at 4° C. for 10 minutes.
10) Centrifugation was performed at 12000 g for 10 minutes. The supernatant was discarded, and 1 ml of PBS was added for resuspension.
11) Centrifugation was performed at 12000 g for 5 minutes. The precipitate was discarded, and the supernatant was transferred to a new 1.5 ml EP tube.
12) Centrifugation was repeated at 12000 g for 5 minutes, and the supernatant was transferred to a new 1.5 ml EP tube, thereby obtaining the original phage library.
13) 10 μl of the precipitate was added to 90 μl of 2YT medium, labeled as 10⁻¹, and serially 10-fold diluted to 10⁻⁹. 20 μl of the gradient diluted samples from the 10⁻⁷, 10⁻⁸, and 10-9 were added to 200 μl of pre-prepared ER2738 with an OD600 of 0.5. The mixture was thoroughly mixed and placed in 37° C. water bath for 10 minutes. Each 108 μl of the mixture was spread onto an LB-AMP solid plate and incubated overnight at 37° C. The number of plaques was counted the next day to determine the titer.
14) Calculation of titer: Plates with a plaque number between 30-300 were selected. Titer was obtained by taking the average of two plates, multiplying the plaque number by the dilution fold and then multiplying by 100.

### 8. Panning of the Phage Display Library

The solid-phase panning process for the phage display library was as follows:

ELISA plates were coated with the target protein, after several wash steps, the recombinant phages bound to the immobilized antigen were eluted using TEA and amplified. After 3-4 rounds of panning, monoclonal colonies were selected for sequencing.
1) The antigen was diluted with PBS to 50 μg/ml, and 150 μl/well of antigen was added to coat a total of 3 wells. The plates were incubated overnight at 4° C.;
2) The target protein was removed, and the wells were blocked with 3% MPBS for 1 hour at room temperature;
3) The lib phage or the precipitate from the previous round of amplification was diluted with 450 μl of 3% MPBS. 6×10¹¹ Pfu of phage was added to the wells coated with control protein (2×10¹¹ Pfu per well). 150 μl of the diluted phage was added to each well and incubated for 1 hour at room temperature;
4) The MPBS in target protein wells was discarded, and the phage from control wells was transferred to the wells coated with target protein for incubation for 1-1.5 hours at room temperature;
5) The phage was discarded, and the wells were washed 8-10 times with 0.05% PBST for 2-3 minutes each time, followed by 4-5 washes with PBS for 2-3 minutes each time. Simultaneously, the 1.5 ml EP tubes that had been blocked previously were washed with PBS;
6) The phages were eluted using 1×TEA at 200 μl per well for 6-8 minutes. The eluted products were collected into the pre-blocked EP tubes, and 100 μl of Tris-HCl was added to each well for neutralization.
7) 10 μl of the output product was added to 90 μl of 2YT medium, labeled as 100, and subsequently diluted 10-fold until 10⁻². 20 μl of the gradient diluted samples from 10¹, 10⁰, 10⁻¹, and 10⁻² were added to 200 μl of pre-prepared ER2738 with an OD600 of 0.5 (10¹ referred to directly adding 20 μl of the undiluted product to ER2738). The mixture was thoroughly mixed and placed in a 37° C. water bath for 10 minutes. Each 108 μl of the mixture was spread onto an LB-AMP solid plate and incubated overnight at 37° C. The number of plaques was counted the next day to determine the titer.
8) Titer calculation: Plates with a plaque number between 30-300 were selected. The titer was calculated by taking the average of two plates, multiplying the number of plaques by the dilution fold and the elution volume.

The cell-based panning process for the phage display library was conducted as follows:

The recombinant cell lines overexpressing the target protein were used. The phage library was sequentially incubated with empty cells and cells overexpressing the target protein. After several washes to remove non-specifically binding phages, the recombinant phages bound to the cell surface were eluted by glycine or TEA and then amplified. After 3-4 rounds of panning, monoclonal colonies were selected for ELISA detection.
1) 1.5 ml EP tubes were blocked with 1% PBSA one day in advance, and left at 4° C. overnight;
2) 1×10⁷ of target cells and control cells were taken, washed three times with PBS, resuspended in 10 mL of 1% PBSA, and placed on a decolorization shaker for low-speed blocking at room temperature for 1 hour, respectively;
3) 1×10¹¹ of phages were added to the control cells and incubated for 1 hour, while the target cells were continued to be blocked;
4) The incubated cells were centrifuged at 1000 g for 5 minutes. The supernatant of the target cells (1% PBSA) was discarded, and the supernatant of the control cells was carefully aspirated and added to the target cell tube. After resuspension, the cells were placed on a shaker for low-speed incubation for 1 hour;
5) The target cell was centrifuged at 1000 g for 5 minutes (while 1.5 ml EP tubes blocked one day in advance were washed three times with PBS). The supernatant of the target cells was discarded, 4 ml of PBS was added for resuspension, and the suspension was distributed into closed 1.5 ml EP tubes. The cells were centrifuged and washed five times with PBS, then transferred to four additional EP tubes for further 5 washes. Finally, the cells were transferred to the same EP tube;
6) The cells were resuspended in 200 μl of PBS, and then 200 μl of 2×TEA was added and immediately blown until the solution was not viscous. 200 μl of Tris-HCl was then added for neutralization, thereby obtaining the final product.
7) The titer of the Output library was determined by the same protocol as for solid-phase panning.

### 9. Phage ELISA

1) 2YT-Amp medium was dispensed into a 96-well deep-well plate with 500 μl per well. Mono clones from the output plate were picked into the wells and cultured at 37° C., 225 rpm until the OD600 reached 0.5. The last two wells, H11 and H12, were filled with only medium, serving as blank controls without clones;
2) Concurrently, the ELISA plate was coated with antigen using CBS at a concentration of 1 μg/ml, with 100 μl per well, and incubated at 37° C. for 2 hours.
3) 2YT-Amp medium was dispensed into an additional 96-well deep-well plate with 500 μl per well. 10 μl of bacterial suspension with OD600 of 0.5 was transferred into the newly dispensed 96-well plate by using a multi-channel pipette, and cultured overnight at 37° C., 225 rpm. This bacterial suspension was for sample sequencing;
4) M13KO7 was added to the bacterial suspension with OD600 of 0.5, mixed thoroughly, and allowed to stand at 37° C. for 15 minutes;
5)

$$The volume of M 13 KO 7 = 10 \times volume \times OD 600 \times 5 \times 108 / M 13 KO 7 titer .$$

6) The bacterial suspension after infection was placed on a shaker and cultured at 37° C., 225 rpm for 45 minutes.
7) The bacterial suspension was centrifuged at 4000 rpm for 10 minutes in a centrifuge. The supernatant was discarded, and the bacteria were resuspended in 800 μl per well of 2YT-AK medium. The plate was then placed back on the shaker and cultured overnight at 30° C., 210 rpm.
8) Concurrently, the antigen in the ELISA plate was removed, and the plate was washed three times with PBST wash buffer and blocked with 250 μl per well of 3% MPBS at 4° C. overnight. An additional blank plate was also blocked as a blank control.
9) On the second day, the 96-well deep-well plate was centrifuged at 4000 rpm for 10 minutes in a centrifuge. The milk in the ELISA plate was discarded, and the plate was washed four times with 200 μL PBST. 50 μl of PBST was added to each well, followed by the addition of 50 μL of centrifuged phage supernatant corresponding to each well. The plate was incubated at 4° C. for 1 hour. The supernatant was discarded, and the plate was washed five times with PBST. HRP-Anti M13 secondary antibody was diluted with PBST and added to each well at 100 μl. After incubation at 4° C. for 45 minutes, the secondary antibody was washed away. The plate was washed five times with PBST. TMB was added for color development at room temperature for 10 minutes, followed by termination with hydrochloric acid. The plate was read, and clones with high S/N ratios were selected for sequencing using the preserved bacterial suspension.

### 10. Construction of Eukaryotic Expression Vectors

Based on the Elisa detection results of phage mono clones, positive clones were selected for sequencing to obtain VHH antibody sequences. The obtained VHH antibody sequences were analyzed and synthesized, and subcloned in tandem with human IgG1Fc into the expression vector pcDNA3.4-hIgG1-Fc2. After the vectors were sequenced and confirmed to be correct, endotoxin-free plasmids were prepared by using Qiagen plasmid extraction kit for later use.

### 11. Construction of Divalent Antibody Expression Vectors

The three candidate antibodies were randomly combined to construct bivalent antibodies, which were expressed in the forms of 2-C10-(G4S)3-2-F3, 2-C10-(G4S)3-2-B1, and 2-F3-(G4S)3-2-B1. Gene synthesis was performed respectively for each of these bivalent antibodies, and they were then subcloned in tandem with human IgG1Fc into the expression vector pcDNA3.4-hIgG1-Fc2. After the vectors were sequenced and confirmed to be correct, endotoxin-free plasmids were prepared by using Qiagen plasmid extraction kit for later use.

### 12. Expression of Single Domain Antibodies

LVTransm transfection reagent and antibody expression vector pcDNA3.4-hIgG1-Fc2 were taken from the refrigerator, thawed at room temperature, and then blown up and down with a pipette until well mixed. PBS buffer was taken out and warmed to room temperature. 500 μL of PBS was taken into one well of a 24-well plate and added with 4 μg pcDNA3.4-hIgG1-Fc2, blown up and down with a pipette until well mixed, then added with 12 μL of LVTransm and immediately blown up and down with a pipette until well mixed. Then the plate was placed at room temperature for 10 minutes. The mixture here was called DNA/LVTransm complex.

The above 532 μL of DNA/LVTransm complex was added into 1.5 mL of 293F cells, gently shaken until well mixed. The cells were placed in a 37° C., 5% CO₂ incubator and cultured at 130 RPM for 6-8 hours. Then 1.5 mL of fresh 293 culture medium was added, and the cells were put back to the incubator for further cultivation.

After 3 days of continuous culture, the culture supernatant was collected and filtered with a 0.45 μm filter membrane, and the filtrate was transferred to a sterile centrifuge tube for following flow cytometry and ELISA detection.

### 13. Flow Cytometry Detection of Recombinant Antibodies Binding to Target Proteins

CHO-K1 cells and CHO-K1-PD1 cell strains were thawed from liquid nitrogen and the cell state was adjusted to logarithmic growth phase. Each of the two kinds of cells was divided into several sections with 5*10\hat{ }5 cells in each section. The expressed antibodies were incubated with target cells respectively by mixing well, and incubating at room temperature for 1 hour. The cells were centrifuged at 800×g at room temperature for 5 minutes, the supernatant containing antibodies was discarded, and the cells were washed with PBS for 3 times. 1 μl of PE-labeled Anti-Human IgG was added, mixed well, and incubated at room temperature in dark for 30 minutes. The cells were centrifuged at 800×g at room temperature for 5 minutes, the supernatant containing the secondary antibodies was discarded, and the cells were washed with PBS for 3 times. The cells were resuspended with 500 μL of PBS, and then subjected to flow cytometry.

### 14 Expression and Purification of Single Domain Antibodies

LVTransm transfection reagent and single chain antibody expression vectors were taken from the refrigerator, thawed at room temperature, and then blown up and down with a pipette until well mixed. PBS or HBSS buffer was taken out and warmed to room temperature. 2 mL of PBS was taken into one well of a 6-well plate and added with 130 μg pcDNA3.4-hIgG1-Fc2, blown up and down with a pipette until well mixed, then added with 400 μL of LVTransm and immediately blown up and down with a pipette until well mixed. Then the plate was placed at room temperature for 10 minutes.

The above DNA/LVTransm complex was added into 50 mL of 293F cells, gently shaken until well mixed. The cells were placed in a 37° C., 5% CO₂ incubator and cultured at 130 RPM for 6-8 hours. Then 50 mL of fresh 293 culture medium was added, and the cells were put back to the incubator for further cultivation.

After 7 days of continuous culture, the culture supernatant was collected and filtered with a 0.45 μm filter membrane, and the filtrate was transferred to a sterile centrifuge tube. The antibody was purified using a Protein A column.

### 15. Detection of PD1-PDL1 Blocking Activity

The Jurkat-PD1-NFAT-Luc reporter gene cell line and aAPCCHO-PDL1 cells were thawed and continuously passaged until logarithmic growth phase. In a 96-well plate, effector cells (Jurkat-PD1-NFAT-Luc) were inoculated at 2×10\hat{ }4 cells per well, and target cells (aAPCCHO-PDL1) were added at a ratio of 1:1. Gradient-diluted antibodies to be tested (positive antibody: Nivolumab; antibodies to be tested) were added to corresponding wells, with a 3-fold gradient dilution applied for a total of 9 dilutions which resulted in final concentrations of 30 μg/mL, 10 μg/mL, 3.333 μg/mL, 1.111 μg/mL, 0.3704 μg/mL, 0.1235 μg/mL, 0.04115 μg/mL, 0.01372 μg/mL, and 0.004572 μg/mL. After co-culturing for 18 hours, 25 μL of One-Glo reagent was added to each well, and the luciferase activity in the wells was measured using a Tecan M1000pro microplate reader.

### 16. Affinity Detection of Recombinant Antibody

PD1-Fc recombinant protein was immobilized on a CM5 chip using 10 mM Acetate buffer, and the prepared single domain antibodies were used as the mobile phase to detect the binding ability of the candidate single domain antibodies to the target protein PD1.

### Example 1: Preparation and Purification of PD1 Single Domain Antibodies

### 1.1 Detection of PD1-Fc Recombinant Protein Activity

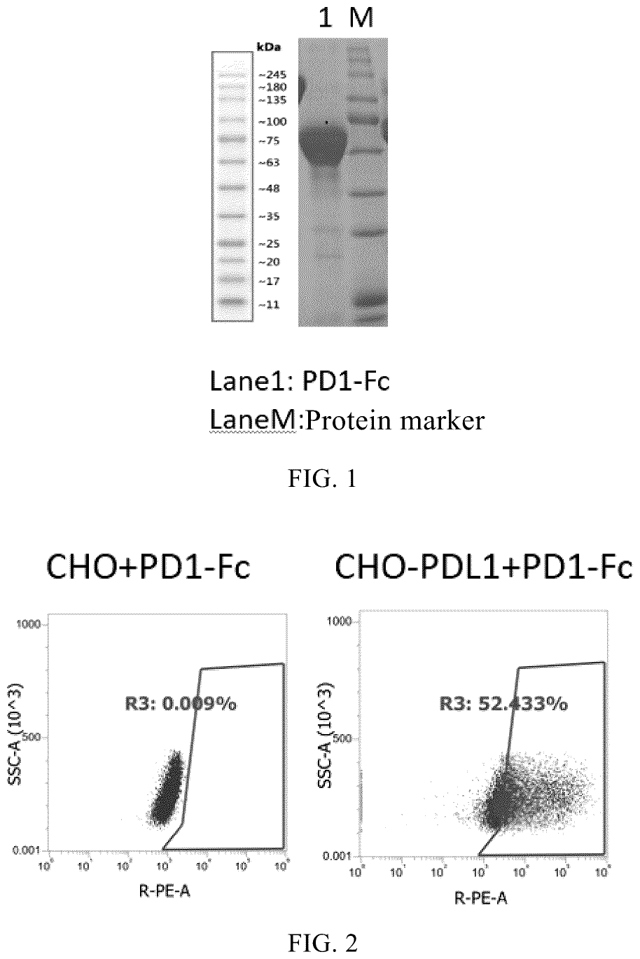

PD1-Fc antigen was prepared through Experimental Method 1 described above, and the expression of PD1-Fc protein was detected by SDS-PAGE. The results are shown in FIG. 1.

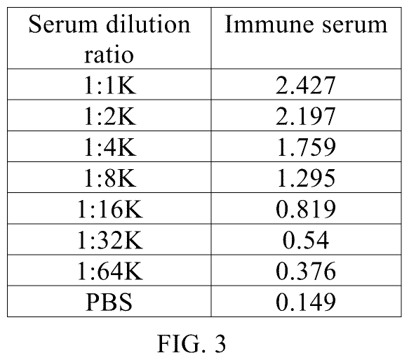

CHO-PDL1 cells were incubated with PD1-Fc, and APC-anti human Fc was used as the secondary antibody. According to the flow cytometry results (FIG. 2), PD1-Fc can bind to PDL1, indicating its activity and suitability for llama immunization and antibody screening.

### 1.2 Detection of Immunological Potency by ELISA

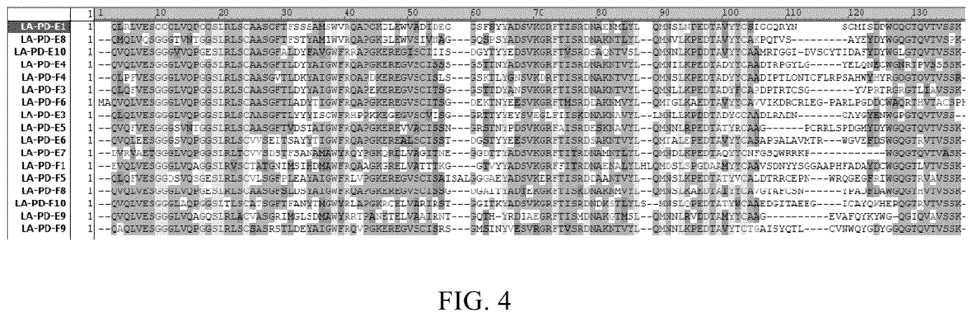

Llamas were immunized through Experimental Method 2 described above. The immunological potency was detected through Experimental Method 3. Specifically, serum was isolated from the immunized llamas, diluted according to the dilution gradient shown in FIG. 3, and subjected to ELISA detection using 96-well plates pre-coated with PD1-His antigen (Sino Biological Inc., Cat #10377-H08H).

Results: As shown in FIG. 3, the ELISA detection results showed that the PD1 immunological potency was high. A boost immunization was conducted, and 150 mL of peripheral blood was collected 10 days later for the construction of a phage display library.

### 1.3 Amplification of VHH Fragments

PBMC isolation and VHH fragment amplification were performed through Experimental Methods 4 and 5 described above. Specifically, peripheral blood was collected from the llamas immunized in Example 1.2, and total RNA thereof was provided. After reverse transcription into cDNA, two rounds of PCR were conducted using primers specific for single domain antibody amplification. The PCR products were analyzed by agarose gel electrophoresis. The first round of PCR yielded PCR bands of approximately 1000 bp and 750 bp, and the 750 bp fragment was recovered and used as the template for the second round of PCR. The second round of PCR yielded a band of approximately 450 bp, representing the VHH fragment.

### 1.4 Diversity Analysis of the Phage Display Library

The construction and diversity analysis of the phage display library were carried out through Experimental Method 6 described above. Specifically, the VHH fragments obtained in Example 1.3 were digested with SfiI enzyme and subcloned into the phage display vector pComF. The vectors were then electrotransformed into SS320 E. coli competent cells to construct the single domain antibody phage display library. 20 mono clones were randomly selected for sequencing to analyze the diversity of the phage display library.

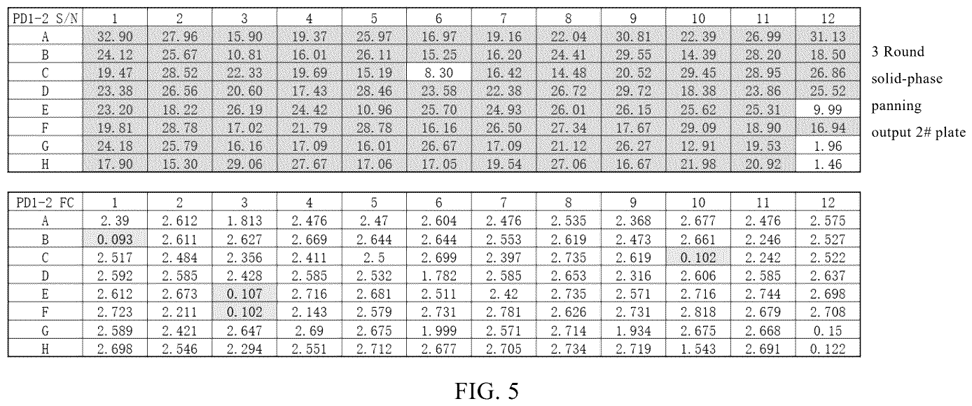

Results: As shown in FIG. 4, sequence alignment revealed that the empty vector rate and antibody repetition rate of the phage display library were no higher than 10%.

### 1.5 Solid-Phase Panning of the Phage Display Library and Identification of Products by Phage ELISA

Solid-phase panning of the phage display library was performed through Experimental Method 7 described above. Specifically, coating with PD1-Fc and Fc recombinant proteins respectively, and the constructed phage display library underwent four rounds of screening and enrichment to enrich positive clones.

1.6 Phage ELISA of the Products from Solid-Phase Panning of the Phage Display Library

From the enriched phage positive clones in Example 1.5, phage mono clones were selected and identified by Phage ELISA through Experimental Method 8 described above.

Specifically, the second and third round outputs from solid-phase panning were chosen for Phage ELISA experiments. Wells of the plate were coated with PD1-Fc antigen protein respectively for Phage Elisa detection. Control groups were directly blocked wells and Fc-coated wells. Clones with an S/N ratio above 5 and not binding to Fc were selected for sequencing and analysis of sequence antibodies.

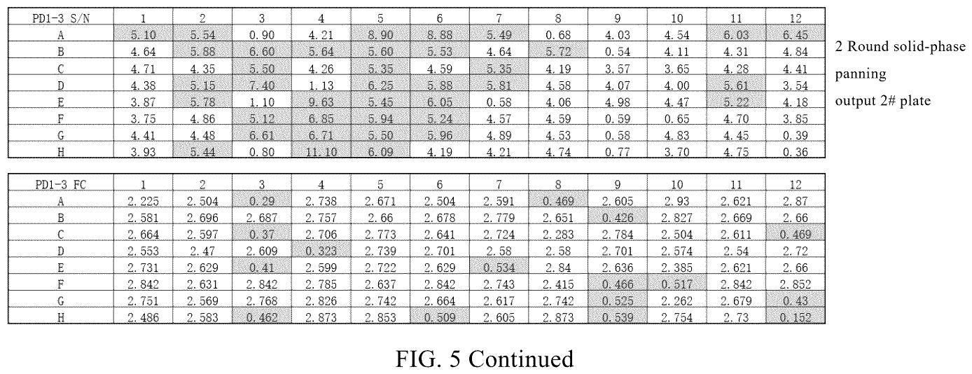

Results: As shown in FIG. 5, a total of four different antibody sequences were obtained through solid-phase panning. The amino acid sequences of their VHH chains and CDRs are presented in Tables 8 and 9, which were used for subsequent construction of eukaryotic expression vectors, transient transfection and expression, and flow cytometric analysis.

Due to the use of solid-phase screening, most of the obtained clones were Fc-positive. Subsequent experiments were arranged to crossly perform cell-based and solid-phase panning to eliminate the interference of Fc antibodies.

TABLE 8
Sequences of the VHH chains of single domain
antibodies of the present invention

| SEQ | | |
|---|---|---|
| ID | | |
| NO: | Name | Sequence |
| 1 | 2-B1 VHH | QVQLVESGGGLVQPGGSLTVSCAASGFSLDAY |
| | | TIGWFRQAPGKEREGIICISRGGRITNY AHSVKD |
| | | RFTISRDGAKNTVYLHMNSLKPEDTGAYYCAA |
| | | VQRDPFICSGYTQSYDHWGQGTQVTVSS |
| 2 | 2-F3 VHH | QVKLEESGGGLVQPGGSLTVSCVASGFSLDAY |
| | | TIGWFRQAPGKEREGIICISRGGRITNYAHSVKD |
| | | RFTISRDGAKNMVYLHMNSLKPEDTGAYYCAA |
| | | VQRDPFICSGYTQSYDLWGQGTQVTVSS |
| 3 | 2-C10 VHH | QVQLVESGGGLVQPGGSLRLSCAASGFSLDAY |
| | | TIGWFRQAPGQEREGISCISRSGRITNYARSAKD |
| | | RFTISRDNAKNTVYLEMKNLRPEDTGAYYCAA |
| | | VEMDPFLCSGYTQSYDQWGQGTQVTVSS |
| 4 | 3-H6 VHH | QLKVVESGGGLVQPGESLRLSCAASGFTLGNY |
| | | GVAWFRQAPGKEREGLSCITSGSTSVTHADSVK |
| | | GRFSVSRDSDKKMVYLQMDSLKPEDTGIYYCA |
| | | AADGGYVCSRNVNANEFDYWGQGTQVTVSS |

CDR regions are underlined in the sequences.

TABLE 9
Sequences of CDR regions of VHH chains
of each antibody

| | | | SEQ |
|---|---|---|---|
| Name of | | | ID |
| Antibody | CDR | Sequence | NO: |
| 2-B1 | CDR1 | GFSLDAYT | 5 |
| | CDR2 | ISRGGRIT | 6 |
| | CDR3 | AAVQRDPFICSGYTQSYDH | 7 |
| 2-F3 | CDR1 | GFSLDAYT | 5 |
| | CDR2 | ISRGGRIT | 6 |
| | CDR3 | AAVQRDPFICSGYTQSYDL | 8 |
| 2-C10 | CDR1 | GFSLDAYT | 5 |
| | CDR2 | ISRSGRIT | 9 |
| | CDR3 | AAVEMDPFLCSGYTQSYDQ | 10 |
| 3-H6 | CDR1 | GFTLGNYG | 11 |
| | CDR2 | ITSGSTSV | 12 |
| | CDR3 | AAADGGYVCSRNVNANEFDY | 13 |

### 1.7 Cross-Performance of Cell-Based and Solid-Phase Panning for the Phage Display Library

Cell-based and solid-phase panning were cross-performed for the phage display library through Experimental Method 8 described above. Specifically, CHO and CHO-PD1 cells along with PD1-Fc antigen were used to perform five rounds of screening and enrichment on the constructed phage display library, thereby enriching positive clones.

1.8 Phage ELISA of Products from Cross-Panning of Solid-Phase and Cell-Based Panning of the Phage Display Library

From the enriched phage positive clones in Example 1.7, phage mono clones were selected and identified by Phage ELISA through Experimental Method 9 described above.

Specifically, Wells of the plate were coated with PD1-Fc and Fc antigen proteins respectively for Phage Elisa detection. Control group was directly blocked wells. The ratio of the ELISA detection results of the antigen group to that of the control group was calculated, and clones with higher ratios were selected for sequencing to analyze the antibody sequences.

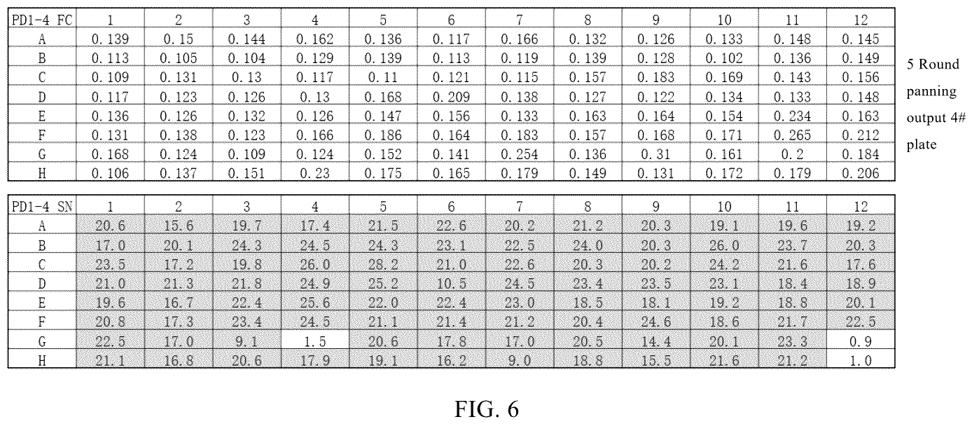

Results: As shown in FIG. 6, the cross-panning of cell-based and solid-phase panning successfully eliminated the interference of Fc antibodies and screened antibodies that specifically bound to PD-1. In this experiment, the antibody sequences were highly enriched, and one antibody sequence was obtained, which was identical to the 2-C10 antibody sequence obtained from solid-phase panning.

### 1.9 Detection of Eukaryotic Expression and Binding of Single Domain Antibody

In this example, vector construction and expression detection were performed on the 4 VHH antibody sequences screened and sequenced in Example 1.6. CMV promoter, signal peptide, and human IgG1 Fc tag were added to the N- and C-terminus of the candidate antibody sequences through Overlap PCR. The purified PCR products were transiently transfected into 293 cells to express the antibodies for flow cytometry detection.

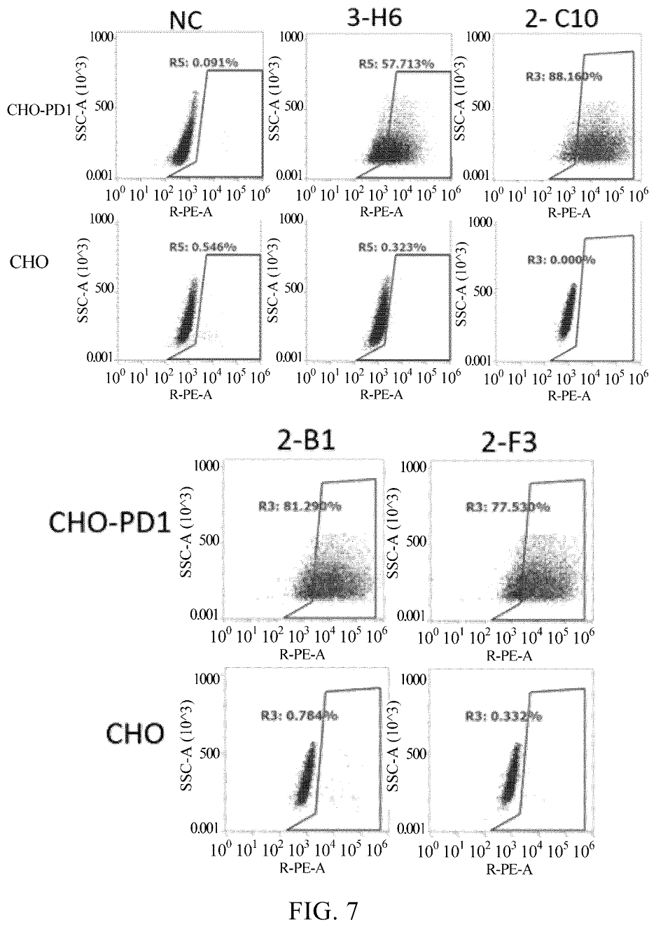

Results: The FACS detection results are shown in FIG. 7, indicating that 3-H6, 2-C10, 2-B1, and 2-F3 clones obtained from phage display library panning are all PD1-specific binding antibodies. The antibodies were expressed and purified for subsequent validation of blocking function.

### Example 2: Detection of Antibody Function

### 2.1 Detection of PD1-PDL1 Blocking Activity (Monovalent Antibody)

The PD1-PDL1 blocking activity was tested using Jurkat-PD1-NFAT-Luc reporter gene cell line and aAPCCHO-PDL1 cells.

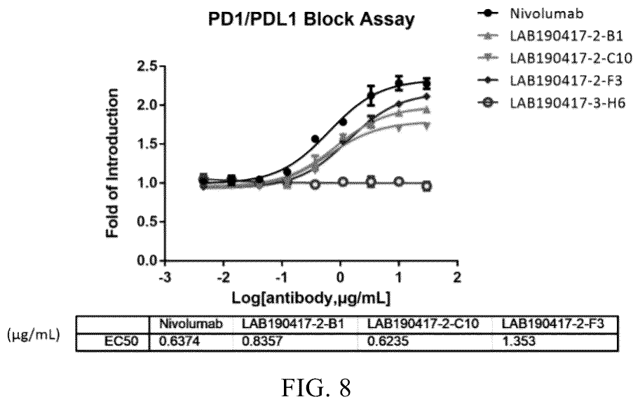

Results: As shown in FIG. 8, the Nivolumab positive control was able to block the interaction between PD1 and PDL1, with an EC50 of 0.6374 μg/mL. Among the tested antibodies, the PD1 single domain antibodies LAB190417-2-B1, LAB190417-2-C10, and LAB190417-2-F3 were all able to block the PD1/PDL1 interaction.

### 2.2 Detection of PD1-PDL1 Blocking Activity (Bivalent Antibody)

To enhance the blocking activity of the antibodies, in this example, the 3 antibodies were randomly combined to construct bivalent antibodies and their blocking activities were tested.

The candidate three antibodies were randomly combined to construct bivalent antibodies with the expression formats of 2-C10-(G4S)3-2-F3, 2-C10-(G4S)3-2-B1, and 2-F3-(G4S)3-2-B1. After purification of the antibodies, the Jurkat-PD1-NFAT-Luc reporter gene cell line and aAPCCHO-PDL1 cells were used to test the PD1-PDL1 blocking activity.

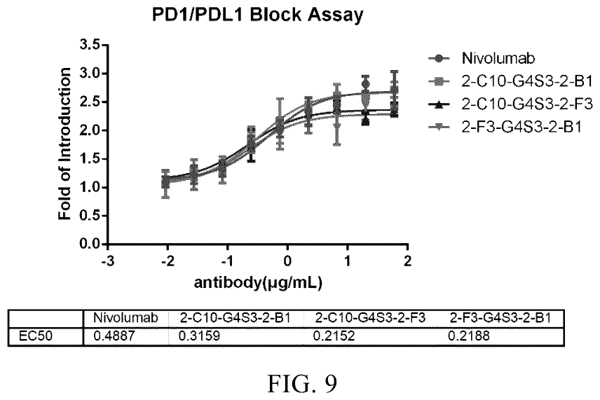

Results: As shown in FIG. 9, the Nivolumab positive control was able to block the PD1/PDL1 interaction, with an EC50 of 0.4887 μg/mL. All the tested bivalent PD1 antibodies were able to block the PD1/PDL1 interaction, wherein 2-C10-(G4S)3-2-B1 exhibited the highest blocking activity.

This example demonstrates that the constructed bivalent antibodies possess highly effective blocking activity against the PD1/PDL1 interaction.

### 2.3 Detection of Affinity of Recombinant Antibodies

PD1-Fc recombinant protein was immobilized on a CM5 chip using 10 mM Acetate buffer, and the prepared bivalent single domain antibodies were used as the mobile phase to detect the binding ability of the candidate single domain antibodies to the target protein PD1.

Results: As shown in Table 10.

TABLE 10
Detection Results of Recombinant Antibody Affinity

| | ka | kd | KD |
|---|---|---|---|
| Nivolumab | 3.987 × 10⁶ M⁻¹s⁻¹ | 8.747 × 10⁻⁵ s⁻¹ | 2.194 × 10⁻¹¹ M |
| 2-C10- | 1.562 × 10⁶ M⁻¹s⁻¹ | 3.289 × 10⁻⁵ s⁻¹ | 2.106 × 10⁻¹¹ M |
| (G4S)₃-2-B1 | | | |
| 2-C10- | 3.181 × 10⁵ M⁻¹s⁻¹ | 8.918 × 10⁻⁵ s⁻¹ | 2.804 × 10⁻¹⁰ M |
| (G4S)₃-2-F3 | | | |
| 2-F3- | 4.183 × 10⁵ M⁻¹s⁻¹ | 1.180 × 10⁻⁴ s⁻¹ | 2.822 × 10⁻¹⁰ M |
| (G4S)3-2-B1 | | | |

This example demonstrates that the three bivalent antibodies of this invention possess high affinity to PD-1, wherein 2-C10-(G4S)3-2-B1 exhibits higher affinity than the existing positive control Nivolumab.

All documents mentioned in the present invention are incorporated by reference herein as if each document were incorporated separately by reference. Furthermore, it should be understood that after reading the foregoing teachings of the invention, various changes or modifications may be made to the invention by those skilled in the art and that these equivalents are equally within the scope of the claims appended to this application.

1. An anti-PD-1 single domain antibody, wherein the complementarity determination regions (CDRs) of the VHH chain of the anti-PD-1 single domain antibody is one or more selected from the group consisting of:
(1) CDR1 as shown in SEQ ID NO: 5, CDR2 as shown in SEQ ID NO: 9, and CDR3 as shown in SEQ ID NO: 10;
(2) CDR1 as shown in SEQ ID NO: 5, CDR2 as shown in SEQ ID NO: 6, and CDR3 as shown in SEQ ID NO: 8;
(3) CDR1 as shown in SEQ ID NO: 5, CDR2 as shown in SEQ ID NO: 6, and CDR3 as shown in SEQ ID NO: 7; and
(4) CDR1 as shown in SEQ ID NO: 11, CDR2 as shown in SEQ ID NO: 12, and CDR3 as shown in SEQ ID NO: 13.

2. The anti-PD-1 single domain antibody according to claim 1, wherein the amino acid sequence of the VHH chain of the anti-PD-1 single domain antibody is selected from sequences shown in SEQ ID NOs: 1, 2, 3 and 4.

3. The anti-PD-1 single domain antibody according to claim 1, wherein the anti-PD-1 single domain antibody is a monomer, a bivalent or a multivalent.

4. The anti-PD-1 single domain antibody according to claim 1, wherein the anti-PD-1 single domain antibody is a bivalent and has a structure shown in Formula I from N-terminus to C-terminus: 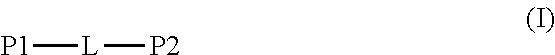
wherein,
“-” is a peptide bond;
L is a linker peptide;
P1 and P2 are each independently a VHH chain.

5. The anti-PD-1 single domain antibody according to claim 4, wherein the sequence of L is (G4S)n, wherein n is a positive integer such as 1, 2, 3, 4, 5 or 6, and preferably n=3.

6. The anti-PD-1 single domain antibody according to claim 4, wherein
P1 has a CDR1 as shown in SEQ ID NO: 5, a CDR2 as shown in SEQ ID NO: 9, and a CDR3 as shown in SEQ ID NO: 10, and P2 has a CDR1 as shown in SEQ ID NO: 5, a CDR2 as shown in SEQ ID NO: 6, and a CDR3 as shown in SEQ ID NO: 7; or
P1 has a CDR1 as shown in SEQ ID NO: 5, a CDR2 as shown in SEQ ID NO: 9, and a CDR3 as shown in SEQ ID NO: 10; and P2 has a CDR1 as shown in SEQ ID NO: 5, a CDR2 as shown in SEQ ID NO: 6, and a CDR3 as shown in SEQ ID NO: 8; or
P1 has a CDR1 as shown in SEQ ID NO: 5, a CDR2 as shown in SEQ ID NO: 6, and a CDR3 as shown in SEQ ID NO: 8, and P2 has a CDR1 as shown in SEQ ID NO: 5, a CDR2 as shown in SEQ ID NO: 6, and a CDR3 as shown in SEQ ID NO: 7.

7. The anti-PD-1 single domain antibody according to claim 4, wherein
P1 has an amino acid sequence as shown in SEQ ID NO: 3, and P2 has an amino acid sequence as shown in SEQ ID NO: 1; or
P1 has an amino acid sequence as shown in SEQ ID NO: 3, and P2 has an amino acid sequence as shown in SEQ ID NO: 2; or
P1 has an amino acid sequence as shown in SEQ ID NO: 2, and P2 has an amino acid sequence as shown in SEQ ID NO: 1.

8. An anti-PD-1 antibody, which comprises one or more VHH chain(s) of the anti-PD1 single domain antibody according to claim 1.

9. A polynucleotide encoding a protein of the anti-PD-1 single domain antibody according to claim 1.

10. An expression vector comprising the polynucleotide according to claim 9.

11. A host cell comprising the expression vector according to claim 10.

12. An immunoconjugate which comprises:
(a) the anti-PD-1 single domain antibody according to claim 1; and
(b) a coupling moiety selected from the group consisting of a detectable label, a drug, a cytokine, a radionuclide, an enzyme, a gold nanoparticle/nanorod, a nanomagnetic particle, a viral coat protein or VLP, and a combination thereof.

13. A pharmaceutical composition which comprises:
(a) the anti-PD-1 single domain antibody according to claim 1; and
(ii) a pharmaceutically acceptable carrier.

14. A method for producing an anti-PD-1 single domain antibody, which comprises the steps of:
(a) culturing the host cell according to claim 11 under conditions suitable for producing a single domain antibody, thereby obtaining a culture containing the anti-PD-1 single domain antibody;
(b) isolating or recovering the anti-PD-1 single domain antibody from the culture; and
(c) optionally, purifying and/or modifying the anti-PD-1 single domain antibody obtained in step (b).

15. A method of preventing and/or treating a disease related to PD-1 signaling pathway, which comprises a step of: administering the anti-PD-1 single domain antibody according to claim 1, to a subject in need thereof.

16. A polynucleotide encoding a protein of the anti-PD-1 single domain antibody according to claim 8.

17. A host cell having the polynucleotide according to claim 9 integrated into its genome.

18. A host cell having the polynucleotide according to claim 16 integrated into its genome.

19. An immunoconjugate which comprises:
(a) the anti-PD-1 single domain antibody according to claim 8; and
(b) a coupling moiety selected from the group consisting of a detectable label, a drug, a cytokine, a radionuclide, an enzyme, a gold nanoparticle/nanorod, a nanomagnetic particle, a viral coat protein or VLP, and a combination thereof.
